(19) United States
(12) Patent Application Publication
(10) Pub. No.: US 2006/0216333 A1
(43) Pub. Date: Sep. 28, 2006

(54) Methods related to the treatment of mucosal associated conditions

(76) Inventors: Richard L. Miller, Maplewood, MN (US); David Q. Ma, St. Paul, MN (US)

Correspondence Address: 3M INNOVATIVE PROPERTIES COMPANY, PO BOX 33427, ST. PAUL, MN, 55133-3427, US

(21) Appl. No.: 10/595,117

(22) PCT Filed: Sep. 1, 2004
(86) PCT No.: PCT/US04/28407

§ 371 Date: Feb. 22, 2006

Related U.S. Application Data
(60) Provisional application No. 60/499,607, filed on Sep. 2, 2003.

(51) Int. Cl. A61K 31/4745; A61K 9/70; A61M 31/00

(52) U.S. Cl. 424/443; 514/292; 604/500

(57) ABSTRACT

Using interrupted delivery of IRMs by intermittently applying an IRM to a mucosal surface it is possible to achieve therapeutic levels and durations of cytokine induction, while substantially reducing irritation side effects.

### CROSS-REFERENCE TO RELATED APPLICATION

The present application claims priority to U.S. Provisional Patent Application Ser. No. 60/499,607, filed on Sep. 2, 2003, which is incorporated herein by reference in its entirety.

### BACKGROUND

There has been a major effort in recent years, with significant successes, to discover new drug compounds that act by stimulating certain key aspects of the immune system, as well as by suppressing certain other aspects of the immune system. These compounds, referred to as immune response modifiers (IRMs), appear to act through basic immune system mechanisms known as toll-like receptors to induce selected cytokine biosynthesis. Also, they may be used to treat a wide variety of diseases and conditions. For example, certain IRMs may be useful for treating viral diseases (e.g., human papilloma virus, hepatitis, herpes), neoplasias (e.g., basal cell carcinoma, squamous cell carcinoma, actinic keratosis), and TH2-mediated diseases (e.g., asthma, allergic rhinitis, atopic dermatitis, multiple sclerosis), and are also useful as vaccine adjuvants. Many of the IRM compounds are small organic molecule imidazoquinoline amine derivatives, but a number of other compound classes are known as well and more are still being discovered. Other IRMs have higher molecular weights, such as oligonucleotides, including CpGs. In view of the great therapeutic potential for IRMs, and despite the important work that has already been done, there is a substantial ongoing need for new means of controlling the delivery and activity of IRMs in order to expand their uses and therapeutic benefits.

### SUMMARY OF THE INVENTION

One problem found when using IRM compounds on mucosal surfaces, e.g., for treatment of mucosal associated conditions, is that it can cause significant irritation or, if low IRM concentrations are used to avoid irritation, can be ineffective. It has now been found, however, that using an interrupted delivery protocol with intermittent application of IRMs can significantly reduce irritation while still achieving therapeutic immune response modulation (i.e., immunomodulation as shown by, e.g., induction of cytokines, stimulation of immune cells, suppression of TH2 immune response, etc.). It appears that limited duration exposure to the IRM compound quickly “jump-starts” the immune response such that a substantial amount of the IRM can then be removed from contact with the mucosal surface to reduce irritation. This will also reduce the risk of systemic exposure via absorption of excess drug. Further, although the IRM imiquimod has been applied and removed before, e.g., using an anal tampon overnight, there was no recognition of the beneficial phenomenon of intermittent application.

The present invention thus relates to methods for reducing irritation by using interrupted delivery (i.e., delivery at intervals such as with a pulsed or periodic delivery) of IRMs by intermittently applying an IRM to a mucosal surface and treatment of mucosal conditions using such delivery protocol. That is, the methods involve applying an IRM at various intervals with removal of the ERM between these intervals such that there is a break between applications. The periods of time between applications, as well as the application times themselves, can vary. That is, the delivery is not necessarily at regular intervals for regular periods of time, although it could be if desired. The periods of application times and breaks are sufficient such that a “jump-starting” of the immune response occurs.

In one particular embodiment, the present invention provides a method of delivering an immune response modifier (IRM) compound to a mucosal surface so as to achieve immunomodulation with reduced irritation. The method includes interrupted delivery of an IRM compound other than imiquimod by intermittently applying the IRM to the mucosal surface and, after each application, removing from the mucosal surface a substantial amount of the IRM at a time before it would otherwise be naturally absorbed or eliminated.

In another embodiment, the present invention provides a method of treating a condition associated with a mucosal surface with an immune response modifier (IRM) compound and reducing irritation caused by the IRM. The method involves interrupted delivery of an IRM other than imiquimod by intermittently applying the MM to the affected mucosal surface for a time sufficient to achieve therapeutic immunomodulation and, after each application, removing from the mucosal surface a substantial amount of the IRM at a time before it would otherwise be naturally absorbed or eliminated.

The term “comprises” and variations thereof do not have a limiting meaning where these terms appear in the description and claims.

As used herein, “a,” “an,” “the,” “at least one,” and “one or more” are used interchangeably.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. Various other features and advantages of the present invention should become readily apparent with reference to the following detailed description, examples, and claims. In several places throughout the specification, guidance is provided through lists of examples. In each instance, the recited list serves only as a representative group and should not be interpreted as an exclusive list.

### DETAILED DESCRIPTION OF ILLUSTRATIVE EMBODIMENTS OF THE PRESENT INVENTION

Although the beneficial effects of IRMs are known, the ability to provide therapeutic benefits via the topical application of an IRM compound to mucosal surfaces for the treatment of mucosal associated conditions is hindered. This is because of the resultant irritation of the mucosal surface that develops with extended contact with an IRM compound and because of undesired systemic delivery of the topically applied IRM compound.

It has now surprisingly been found that the intermittent application of an IRM to a mucosal surface provides a therapeutic benefit without the irritation of the mucosal tissue associated with continuous (or extended) contact with the IRM. Thus, the present invention provides new methods for using IRM compounds to treat or prevent conditions associated with a mucosal surface. In some embodiments, the invention provides methods that are particularly advantageous for the topical application of an IRM to the cervix for treatment of cervical conditions such as cervical dysplasias including dysplasia associated with human papillomavirus (HPV), low-grade squamous intraepithelial lesions, high-grade squamous intraepithelial lesions, atypical squamous cells of undetermined significance (typically, with the presence of high-risk HPV), and cervical intraepithelial neoplasia (CIN).

The present invention provides methods of reducing the irritation of a mucosal surface associated with treating a mucosal associated condition with an IRM. Alternatively stated, the present invention provides methods of delivering an IRM to a mucosal surface so as to achieve immunomodulation with reduced irritation.

The present invention also provides methods of treating a mucosal associated condition. Alternatively stated, the present invention provides methods of treating a condition associated with a mucosal surface with an IRM compound and reducing irritation caused by the IRM.

These methods include intermittently applying an IRM to the mucosal surface. Preferably, after each application a substantial amount of the IRM is removed at a time that is less than the time required for the same amount of the IRM (i.e., the amount that is removed) to be naturally absorbed or eliminated. Preferably, after each intermittent application a substantial amount of the IRM is removed less than 8 hours after it is applied.

Preferably, a substantial amount of the IRM is removed with the same device used to apply the IRM. That is, it is not removed by a method, such as, for example, douching.

In certain embodiments, the IRM is predispersed within a solid matrix capable of releasing the IRM. The IRM may be removed with the same solid predispersed matrix used to apply the IRM. Also, for such methods, a substantial amount of the IRM may be removed at a time period that is less than 8 hours after it is applied.

In certain embodiments, the invention provides a method of treating a papilloma virus infection of the cervix using intermittent application of an IRM. In certain other embodiments, the invention provides a method of treating atypical squamous cells of undetermined significance with the presence of high-risk HPV.

Delivery Times:

The methods of the present invention reduce the time that an IRM is in contact with a mucosal surface. A mucosal surface is contacted with an IRM for a period of time sufficient to initiate induction of cytokine production. Then, after a specified delivery time, the IRM is removed from the mucosal surface, reducing the development of mucosal surface irritation. Such removal of the IRM also serves to remove excess IRM. Surprisingly, using intermittent application of an IRM, beneficial results can be obtained by “jump-starting” cytokine production, without the significant irritation to mucosal tissue that can result from conventional application methods.

As used herein, a “specified delivery time” is the time period from the application of the IRM to the removal of a substantial amount of the IRM. As used herein, “substantial amount” means at least 25% and usually at least 50% by weight of the IRM that was originally applied. The specified delivery time for the application of an IRM to a mucosal surface is typically and preferably a time period of less than eight hours. However, the specified delivery time for the application of an IRM to a mucosal surface may be six hours or less, four hours or less, two hours or less, or one hour or less, depending on the desired treatment regimen. The specified delivery time for the application of an IRM to a mucosal surface may be even shorter. For example, it can be sixty minutes or less, thirty minutes or less, or even twenty minutes or less. Typically, the specified delivery time is at least ten minutes, and preferably at least fifteen minutes for desired effect.

In the methods of the present invention, an IRM may be applied once a week. In the methods of the present invention, an IRM may also be applied several times a week. For example, an IRM may be applied twice a week, three times a week, or five times a week. An IRM may also be applied daily.

In the methods of the present invention, the applications of an IRM may extend for a total time period of at least one week, at least two weeks, at least three weeks, at least one month, at least two months, at least three months, or more, depending on the desired treatment regime.

The actual dosing (treatment) regimen used for a given condition or subject may depend at least in part on many factors known in the art, including, but not limited to, the physical and chemical nature of the IRM compound, the nature of the delivery material, the amount of IRM being administered, the state of the subject's immune system (e.g., suppressed, compromised, stimulated), the method of administering the IRM, and the species to which the IRM is being administered.

The methods of the present invention may be applicable for any suitable subject. Suitable subjects include, but are not limited to, animals such as, but not limited to, humans, non-human primates, rodents, dogs, cats, horses, pigs, sheep, goats, cows, or birds.

The methods of the present invention are suitable for a variety of medical objectives, including therapeutic, prophylactic (e.g., as a vaccine adjuvant), or diagnostic. As used herein, “treating” a condition or a subject includes therapeutic, prophylactic, and diagnostic treatments.

The term “an effective amount” (e.g., therapeutically or prophylactically) means an amount of the compound sufficient to induce a desired (e.g., therapeutic or prophylactic) effect, such as cytokine induction, inhibition of TH2 immune response, antiviral or antitumor activity, reduction or elimination of neoplastic cells. The amount of an IRM compound that will be therapeutically effective in a specific situation will depend on such things as the activity of the particular compound, the dosing regimen, the application site, the particular formulation and the condition being treated. As such, it is generally not practical to identify specific administration amounts herein; however, those skilled in the art will be able to determine appropriate therapeutically effective amounts based on the guidance provided herein and information available in the art pertaining to these compounds.

Mucosal Associated Conditions:

The methods of the present invention may be used for the application of an IRM compound to a mucosal surface for the treatment of a mucosal associated condition. The methods of the present invention are particularly advantageous for the mucosal application of an IRM for a period of time sufficient to obtain a desired therapeutic effect without the same level of undesired irritation that can develop after the continuous (or extended) exposure of a mucosal surface to an IRM. The methods of the present invention are also advantageous to obtain a desired therapeutic effect from the mucosal application of an IRM while reducing the undesired systemic absorption of the IRM.

As used herein, a “mucosal associated condition” means an inflammatory, infectious, neoplastic, or other condition that involves a mucosal surface or that is in sufficient proximity to a mucosal tissue to be affected by a therapeutic agent topically applied to the mucosal tissue surface. Examples of such conditions include a papilloma virus infection of the cervix, cervical dysplasias including dysplasia associated with human papillomavirus (HPV), low-grade squamous intraepithelial lesions, high-grade squamous intraepithelial lesions, atypical squamous cells of undetermined significance (typically, with the presence of high risk HPV), and cervical intraepithelial neoplasia, an atopic allergic response, allergic rhinitis, a neoplastic lesion, and a premalignant lesion.

As used herein, a “mucosal surface” includes mucosal membranes such as buccal, gingival, nasal, ocular, tracheal, bronchial, gastrointestinal, rectal, urethral, ureteral, vaginal, cervical, and uterine mucosal membranes. For example, one could treat oral lesions, vaginal lesions, or anal lesions by the methods described. One could also use the methods in combination with mucosal application of vaccines. Depending on the IRM concentration, formulation composition, and mucosal surface, the therapeutic affect of the IRM may extend only to the superficial layers of the mucosal surface or to tissues deep below the surface.

In one embodiment, an IRM can be applied to vaginal or supravaginal mucosal surfaces for the treatment of a cervical dysplasia. In other embodiments, an IRM can be applied to the mucosal surfaces of the rectum for the treatment of, e.g., anal canal condyloma.

Cervical dysplasias to be treated by the methods of the present invention preferably include dysplastic conditions such as low-grade squamous intraepithelial lesions, high-grade squamous intraepithelial lesions, atypical squamous cells of undetermined significance (typically, with the presence of high-risk HPV), and cervical intraepithelial neoplasia (CIN).

Approximately 16,000 new cases of invasive cancer of the cervix are diagnosed each year in the U.S. despite extensive screening of women to detect predictive cellular changes. There are also about 3,000 deaths due to cervical cancer in the U.S. alone and this is usually secondary to not detecting the primary cancerous lesion in a timely manner.

The Papanicoulaou Test (Pap smear) is the screening test that has been accepted since the 1950s as the method to detect abnormal cells of the cervix, including inflammation and dysplasia, which includes cervical cancer. This screening test has been widely adopted in industrialized countries and has had a profound impact on mortality associated with cervical cancers. An abnormal Pap smear prompts close observation for disease progression with the potential for the therapeutic interventions of destruction or excision of cancerous or pre-cancerous tissues. These excisional treatments are expensive, uncomfortable and associated with failure rates that range from 2% to 23% and with higher failure rates reported for the more advanced lesions. Failure rates have recently been documented to approximate 10% following laser treatment.

The etiologic agent for cervical cancer was originally thought to be the herpes virus. However, there was a gradual shift from this focus on herpes virus to the human papillomavirus (HPV). Improved experimental methods over the recent past nave allowed the characterization of a full spectrum of HPV subtypes, which has resulted in the conclusion that the high risk HPV types (e.g., HPV 16, 18, and less frequently 31, 33, 35, 45) are very likely the exclusive initiating factor (i.e., oncogenic agent) for cervical dysplasia and subsequent cancers. The mechanism of HPV transformation of the normal cell to a dysplastic cell is associated with the HPV encoded oncoproteins (E6 and E7) from the high risk genotypes binding the cell's tumor suppressor gene products p53 and Rb resulting in disruption of the cell cycle control mechanism in which p53 and Rb play an important role. In addition, the application of these molecular methods has resulted in the epidemilogic observation that HPV is isolated from approximately 93% of cervical tumors, which has further strengthened the generally accepted conclusion that HPV infection is the most important initiating agent for cervical cancer.

Exposure to HPV is common in sexually active women, but it does not invariably lead to dysplasia or cancer in most of the exposed women. Infected women who harbor persistent viral DNA have about five times the chance of persistent dysplasia compared to women who are able to eradicate the virus. The importance of cell-mediated immune response to HPV infection is illustrated by the observation that the antibody mediated immune response is not effective in eliminating established infections as is demonstrated by the fact that patients with invasive cervical cancer often exhibit high antibody levels against the viral E6 and E7 proteins. This particular antibody response probably reflects extensive antigen exposure in the face of increasing tumor burden. In contrast to the apparently inconsequential effect of the humoral immune response; the cell-mediated immune response (Th-1-Type Response) appears to be effective in controlling tumor progression. Regression of intraepithelial lesions is accompanied by a cellular infiltrate consisting of CD4⁺ T-cells, CD8⁺ T-cells, natural killer cells (NK) and macrophages. This inflammatory infiltrate was usually associated with tumor regression that is in contrast to women who lack the ability to mount this inflammatory response and who experience disease progression. In addition, patients with a defect in cell-mediated immunity have increased cervical cancer rates, whereas those with defects in the production of antibody do not exhibit the same susceptibility.

Suitable Immune Response Modifiers:

Immune response modifiers (“IRMs”) useful in the methods of the present invention include compounds that act on the immune system by inducing and/or suppressing cytokine biosynthesis. IRMs possess potent immunostimulating activity including, but not limited to, antiviral and antitumor activity, and can also down-regulate other aspects of the immune response, for example, shifting the immune response away from a TH-2 immune response, which is useful for treating a wide range of TH-2 mediated diseases. IRMs can also be used to modulate humoral immunity by stimulating antibody production by B cells. Further, various IRMs have been shown to be useful as vaccine adjuvants (see, e.g., U.S. Pat. Nos. 6,083,505, 6,406,705, and International Publication No. WO 02/24225).

In particular, certain IRMs effect their immunostimulatory activity by inducing the production and secretion of cytokines such as, e.g., Type I interferons, TNF-α, IL-1, IL-6, IL-8, IL-10, IL-12, MIP-1, and/or MCP-1, and can also inhibit production and secretion of certain Th2 cytokines, such as IL-4 and IL-5. Some IRMs are said to suppress IL-1 and TNF (see, e.g., International Patent Publication No. WO 00/09506). Preferred IRMs are so-called small molecule IRMs, which are relatively small organic compounds (e.g., molecular weight under about 1000 daltons, preferably under about 500 daltons, as opposed to large biologic protein, peptides, and the like).

Although not bound by any single theory of activity, some IRMs are known to be agonists of at least one Toll-like receptor (TLR). IRMs that are agonists for TLRs selected from 6, 7, 8, and 9 may be particularly useful for certain applications. Some small molecule IRMs are agonists of TLRs such as 6, 7, and 8, while oligonucleotide IRM compounds are agonists of TLR9, and perhaps others. Thus, in some embodiments, the IRM that is applied to a mucosal surface may be a compound identified as an agonist of one or more TLRs. Preferably, the IRM activates a TLR7.

Preferred IRM compounds comprise a 2-aminopyridine fused to a five membered nitrogen-containing heterocyclic ring. Examples of classes of small molecule IRM compounds include, but are not limited to, imidazoquinoline amines, including but not limited to, substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, and 6-, 7-, 8-, or 9-aryl or heteroaryl substituted imidazoquinoline amines; tetrahydroimidazoquinoline amines, including but not limited to, amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, and thioether substituted tetrahydroimidazoquinoline amines; imidazopyridine amines, including but not limited to, amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; imidazotetrahydronaphthyridine amines; oxazoloquinoline amines; thiazoloquinoline amines; oxazolopyridine amines; thiazolopyridine amines; oxazolonaphthyridine amines; thiazolonaphthyridine amines; and 1H-imidazo dimers fused to pyridine amines, quinoline amines, tetrahydroquinoline amines, naphthyridine amines, or tetrahydronaphthyridine amines, such as those disclosed in, for example, U.S. Pat. Nos. 4,689,338; 4,929,624; 4,988,815; 5,037,986; 5,175,296; 5,238,944; 5,266,575; 5,268,376; 5,346,905; 5,352,784; 5,367,076; 5,389,640; 5,395,937; 5,446,153; 5,482,936; 5,693,811; 5,741,908; 5,756,747; 5,939,090; 6,039,969; 6,083,505; 6,110,929; 6,194,425; 6,245,776; 6,331,539; 6,376,669; 6,451,810; 6,525,064; 6,545,016; 6,545,017; 6,558,951; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; European Patent 0 394 026; U.S. Patent Publication Nos. 2002/0016332; 2002/0055517; 2002/0110840; 2003/0133913; 2003/0199538; and 2004/0014779; and International Patent Publication No. WO 04/058759.

Additional examples of small molecule IRMs said to induce interferon (among other things), include purine derivatives (such as those described in U.S. Pat. Nos. 6,376,501 and 6,028,076), imidazoquinoline amide derivatives (such as those described in U.S. Pat. No. 6,069,149), 1H-imidazopyridine derivatives (such as those described in Japanese Patent Application 9-255926) and benzimidazole derivatives (such as those described in U.S. Pat. No. 6,387,938). 1H-imidazopyridine derivatives (such as those described in U.S. Pat. No. 6,518,265 and European Patent Application EP 1 256 582)) are said to inhibit TNF and IL-1 cytokines.

Examples of small molecule IRMs which comprise a 4-aminopyrimidine fused to a five membered nitrogen-containing heterocyclic ring include adenine derivatives (such as those described in U.S. Pat. Nos. 6,376,501; 6,028,076; and 6,329,381; and in International Patent Publicaton No. WO 02/08595).

In certain embodiments, the methods of the present invention do not use imiquimod. In certain embodiments, the methods of the present invention do not use imiquimod or resiquimod.

In certain embodiments, the immune response modifier is selected from the group consisting of imidazoquinoline amines, tetrahydroimidazoquinoline amines, imidazopyridine amines, 6,7-fused cycloalkylimidazopyridine amines, 1,2-bridged imidazoquinoline amines, imidazonaphthyridine amines, imidazotetrahydronaphthyridine amines, oxazoloquinoline amines, thiazoloquinoline amines, oxazolopyridine amines, thiazolopyridine amines, oxazolonaphthyridine amines, thiazolonaphthyridine amines, 1H-imidazo dimers fused to pyridine amines, quinoline amines, tetrahydroquinoline amines, naphthyridine amines, or tetrahydronaphthyridine amines, and combinations thereof.

In certain embodiments, the methods of the present invention the IRM is selected from the group consisting of amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, 6-, 7-, 8-, or 9-aryl or heteroaryl substituted imidazoquinoline amines, amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, thioether substituted imidazopyridine amines, 1,2-bridged imidazoquinoline amines, 6,7-fused cycloalkylimidazopyridine amines, imidazonaphthyridine amines, tetrahydroimidazonaphthyridine amines, oxazoloquinoline amines, thiazoloquinoline amines, oxazolopyridine amines, thiazolopyridine amines, oxazolonaphthyridine amines, thiazolonaphthyridine amines, pharmaceutically acceptable salts thereof, and combinations thereof.

In certain other embodiments, the IRM is selected from the group consisting of urea substituted imidazoquinoline amines, thioether substituted imidazoquinoline amines, imidazonaphthyridine amines, and pharmaceutically acceptable salts thereof. Preferably, the IRM is an imidazonaphthyridine amine or a pharmaceutically acceptable salt thereof, and more preferably, the IRM is 1-(2-methylpropyl)-1H-imidazo[4,5-c][1,5]naphthyridin-4-amine or a pharmaceutically acceptable salt thereof.

Other IRMs include large biological molecules such as oligonucleotide sequences. Some IRM oligonucleotide sequences contain cytosine-guanine dinucleotides (CpG) and are described, for example, in U.S. Pat. Nos. 6,1994,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG and are described, for example, in International Patent Publication No. WO 00/75304.

IRMs such as imiquimod—a small molecule, imidazoquinoline IRM, marketed as ALDARA (3M Pharmaceuticals, St. Paul, Minn.)—have been shown to be useful for the therapeutic treatment of warts, as well as certain cancerous or pre-cancerous lesions (See, e.g., Geisse et al., J. Am. Acad. Dermatol., 47(3): 390-398 (2002); Shumack et al., Arch. Dermatol., 138: 1163-1171 (2002)).

Other diseases for which IRMs may be used as treatments include, but are not limited to:

viral diseases, such as genital warts, common warts, plantar warts, hepatitis B, hepatitis C, herpes simplex virus type I and type II, molluscum contagiosum, variola, HIV, CMV, VZV, rhinovirus, adenovirus, coronavirus, influenza, para-influenza;

bacterial diseases, such as tuberculosis, and mycobacterium avium, leprosy;

other infectious diseases, such as fungal diseases, chlamydia, candida, aspergillus, cryptococcal meningitis, pneumocystis carnii, cryptosporidiosis, histoplasmosis, toxoplasmosis, trypanosome infection, leishmaniasis;

neoplastic diseases, such as intraepithelial neoplasias, cervical dysplasia, actinic keratosis, basal cell carcinoma, squamous cell carcinoma, hairy cell leukemia, Karposi's sarcoma, melanoma, renal cell carcinoma, myelogeous leukemia, multiple myeloma, non-Hodgkin's lymphoma, cutaneous T-cell lymphoma, and other cancers;

TH-2 mediated, atopic, and autoimmune diseases, such as atopic dermatitis or eczema, eosinophilia, asthma, allergy, allergic rhinitis, systemic lupus erythematosis, essential thrombocythaemia, multiple sclerosis, Ommen's syndrome, discoid lupus, alopecia greata, inhibition of keloid formation and other types of scarring, and enhancing would healing, including chronic wounds; and

As a vaccine adjuvant for use in conjunction with any material that raises either humoral and/or cell mediated immune response, such live viral and bacterial immunogens and inactivated viral, tumor-derived, protozoal, organism-derived, fungal, and bacterial immunogens, toxoids, toxins, polysaccharides, proteins, glycoproteins, peptides, cellular vaccines, DNA vaccines, recombinant proteins, glycoproteins, and peptides, and the like, for use in connection with, e.g., BCG, cholera, plague, typhoid, hepatitis A, B, and C, influenza A and B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, and yellow fever.

IRMs may also be particularly helpful in individuals having compromised immune functioning, such as those with HIV AIDS, transplant patients, and cancer patients.

IRM Formulations:

In the methods of the present invention, an IRM may be provided as a formulation suitable for delivery to a mucosal surface. Suitable formulations can include, but are not limited to, creams, gels, foams, ointments, lotions, solutions, suspensions, dispersions, emulsions, microemulsions, pastes, powders, oils, wipes, or sprays.

The amount or concentration of the IRM is preferably at least 0.001% by weight based on the total formulation weight. The amount or concentration of the IRM is preferably no greater than 10% by weight based on the total formulation weight. In certain embodiments, the amount of the IRM is at least 0.003% by weight, such as, for example, at least 0.005%, at least 0.01%, at least 0.03%, at least 0.10%, at least 0.30%, and at least 1.0%. In other embodiments, the amount of the IRM is at most 5.0% by weight, such as, for example, at most 3.0%, and at most 1.0%. Certain exemplary ranges include, for example, from 0.01% to 5.0% by weight, or from 0.03 to 1.0% by weight.

One or more IRMs may be present in the formulation as the sole therapeutically active ingredient or in combination with other therapeutic agents. Such other therapeutic agents may include, for example, antibiotics, such as penicillin or tetracycline, corticosteroids, such as hydrocortisone or betamethasone, nonsteroidal antiinflammatories, such as fluriprofen, ibuprofen, or naproxen, or antivirals, such as acyclovir or valcyclovir.

IRM formulations for use in the methods of the present invention may include a fatty acid if desired. As used herein, the term “fatty acid” means a carboxylic acid, either saturated or unsaturated, comprising 6 to 28 carbon atoms, such as, for example, from 10 to 22 carbon atoms. Non-limiting examples of such fatty acids include isostearic acid, oleic acid, and linear or branched chained carboxylic acids of 6 to 18 carbon atoms. The fatty acid may be present in the formulation in an amount sufficient to solubilize the IRM compound. In one embodiment, the amount of the fatty acid can range from 1% to 99% by weight based on the total weight of the formulation, such as, for example, from 30% to 70%, from 40% to 60%, and from 45% to 55%. In certain embodiments, the amount of the fatty acid is at least 10% by weight, such as, for example, at least 20%, at least 30%, and at least 40%. In certain embodiments, the amount of the fatty acid is at most 70% by weight, such as, for example, at most 60% and at most 55%. The fatty acid component of the formulation can comprise one or more fatty acids.

IRM formulations may additionally include at least one emollient if desired. Examples of useful emollients include, but are not limited to, fatty acid esters, for example, isopropyl myristate, isopropyl palmitate, diisopropyl dimer dilinoleate; triglycerides, for example, caprylic/capric triglyceride; cetyl esters wax; hydrocarbons of 8 or more carbon atoms, for example, light mineral oil, white petrolatum; waxes, for to example, beeswax; and long chain alcohols, for example, cetyl alcohol and stearyl alcohol. In some embodiments, the emollient is chosen from one or more of isopropyl myristate, isopropyl palmitate, caprylic/capric triglyceride, and diisopropyl dimer dilinoleate. In other embodiments the emollient is isopropyl myristate. In one embodiment, the amount of emollient can range from 1% to 99% by weight based on the total weight of the formulation, such as, for example, from 30% to 70%, from 40% to 60% and from 45% to 55%. In certain embodiments, the amount of the emollient is at least 10% by weight, such as, for example, at least 20%, at least 30%, at least 40%, and at least 45%. In certain embodiments, the amount of the emollient is at most 70% by weight, such as, for example, at most 60% and at most 55%.

Certain preferred formulations include both a fatty acid and a fatty acid ester. For example, isostearic acid and isopropyl myristate can be used together. A particularly preferred formulation includes a 1:1 weight ratio of isostearic acid and isopropyl myristate.

IRM formulations can also include a viscosity enhancing agent if desired. Examples of suitable hydrophilic viscosity enhancing agents include cellulose ethers such as hydroxypropylmethylcellulose, hydroxyethylcellulose, hydroxypropylcellulose, and carboxymethylcellulose; polysaccharide gums such as xanthan gum; and homopolymers and copolymers of acrylic acid crosslinked with allyl sucrose or allyl pentaerythriol such as those polymers designated as carbomers in the United States Pharmacopoeia.

IRM formulations can additionally comprise an emulsifier if desired. Suitable emulsifiers include non-ionic surfactants such as, for example, polysorbate 60, sorbitan monostearate, polyglyceryl-4 oleate, polyoxyethylene(4) lauryl ether, etc. In certain embodiments, the emulsifier is chosen from poloxamers (e.g., POLOXAMER 188, a poly(ethylene glycol)-block-poly(propylene glycol)-block-poly(ethylene glycol), available from BASF, Ludwigshafen, Germany) and sorbitan trioleate (e.g., SPAN 85 available from Uniqema, New Castle, Del.).

In certain embodiments, IRM formulations can also include at least one chelating agent. The chelating agent functions to chelate metal ions that may be present in the formulation. Suitable chelating agents include salts of ethylenediaminetetraacetate (EDTA), such as the disodium salt.

In certain embodiments, IRM formulations can also include one or more preservatives. Examples of suitable preservatives include methylparaben, ethylparaben, propylparaben, phenoxyethanol, iodopropynyl butylcarbamate, sorbic acid, a fatty acid monoester of glycerin such as glycerol monolaurate, and a fatty acid monoester of propylene glycol such as propylene glycol monocaprylate.

IRM formulations may additionally comprise at least one pH adjuster if desired. Suitable pH adjusters include organic bases and inorganic bases such as, for example, KOH and NaOH.

Suitable Delivery Devices:

An IRM may be applied to a mucosal surface with the use of a delivery device. Suitable devices include cervical caps, diaphragms, and solid matrices such as tampons, cotton sponges, cotton swabs, foam sponges, and suppositories. The IRM can be removed by withdrawing the device from contact with the mucosal surface.

In some embodiments the device can be used in combination with an IRM formulation. In one embodiment, a cream or a gel containing an IRM can be placed into the concave region of a cervical cap, which is then place directly over the cervix. In another embodiment, a cotton or foam sponge can be used in combination with a solution containing an IRM.

In some embodiments the IRM or IRM formulation may be predispersed in a matrix. In one embodiment, a cotton or foam sponge can be impregnated with solution containing an IRM prior to the sponge being placed in contact with a mucosal surface. Herein, “predispersed” means that the IRM is substantially uniformly dispersed or distributed throughout the solid matrix, as opposed to merely being applied to the surface of the solid matrix. The IRM can be predispersed in a solid matrix as a solution, a powder, or an emulsion.

In some embodiments, an IRM may be included in an IRM formulation that includes a fatty acid, including isostearic acid. In a preferred embodiment, an IRM may be included in an IRM formulation that includes a fatty acid, for example, isostearic acid, and an emollient, for example isopropyl myristate.

In some embodiments, an applicator may be used to place the device and/or IRM in the proper location on the mucosal surface. Examples of such applicators include, for example, cardboard or plastic tube applicators commonly used for inserting tampons or suppositories.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

In the examples below the serum and intravaginal cytokine data were obtained using the following general test method.

Rats were acclimated to collars (Lomir Biomedical, Malone, N.Y.) around the neck on two consecutive days prior to actual dosing. Rats were collared to prevent removal of the device and ingestion of the drug. Animals were then dosed intravaginally with a removable device or with 50 mL of cream. Single dosed rats received one intravaginal dose with samples collected at various times following dosing. Blood was collected by cardiac puncture. Blood was allowed to clot briefly at room temperature and serum was separated from the clot via centrifugation. The serum was stored at −20° C. until it was analyzed for cytokine concentrations.

Following blood collection, the rats were euthanized and their vaginal tract, including the cervix, was then removed and the tissue was weighed, placed in a sealed 1.8 mL cryovial and flash frozen in liquid nitrogen. The frozen vaginal tissue sample was then suspended in 1.0 mL of RPMI medium (Celox, St. Paul, Minn.) containing 10% fetal bovine serum (Atlas, Fort Collins, Colo.), 2 mM L-glutamine, penicillin/streptomycin and 2-mercaptoethanol (RPMI complete) combined with a protease inhibitor cocktail set III (Calbiochem, San Diego, Calif.). The tissue was homogenized using a Tissue Tearor (Biospec Products, Bartlesville, Okla.) for approximately one minute. The tissue suspension was then centrifuged at 2000 rpm for 10 minutes under refrigeration to pellet the debris, and the supernatant collected and stored at −20° C. until analyzed for cytokine concentrations.

ELISA kits for rat TNF were purchased from BD PharMingen (San Diego, Calif.) and the rat MCP-1 ELISA kits were purchased from BioSource Intl. (Camarillo, Calif.).

Both kits were performed according to manufacturer's specifications. Results for both TNF and MCP-1 are expressed in pg/mL and are normalized per 200 mg of tissue. The sensitivity of the TNF ELISA, based on the lowest value used to form the standard curve, is 63 pg/mL and for the MCP-1 ELISA it is 12 pg/mL. “Post dosing” means after treatment initiation. For example, if a device was inserted a time 0 hours and removed at 2 hours and cytokines were assayed at 4 hours, then the cytokines were assayed at 4 hours post dosing.

The IRM compounds used in the examples are identified in the table below.

| IRM | Chemical Name | Reference |
|---|---|---|
| IRM 1 | 2-propyl[1,3]thiazolo[4,5-c]quinoline-4-amine | U.S. Pat. No. 6,110,929 |
| | | Example 12 |
| IRM 2 | 4-amino-α,α,2-trimethyl-1H-imidazo[4,5-c]quinoline- | U.S. Pat. No. 5,266,575 |
| | 1-ethanol | Example C1 |
| IRM 3 | 1-(2-methylpropyl)-1H-imidazo[4,5- | U.S. Pat. No. 6,194,425 |
| | c][1,5]naphthyridin-4-amine | Example 32 |
| IRM 4 | N-{4-[4-amino-2-(2-methoxyethyl)-1H- | U.S. Pat. No. 6,331,539 |
| | imidazo[4,5-c]quinolin-1- | Example 111 |
| | yl]butyl}methanesulfonamide | |
| IRM 5 | N-[3-(4-amino-2-butyl-1H-imidazo[4,5- | U.S. Pat. No. 6,573,273 |
| | c]quinolin-1-yl)propyl-N′-butylurea | Example 150 |
| IRM 6 | 2-butyl-1-{2-[(1-methylethyl)sulfonyl]ethyl}- | U.S. Pat. No. 6,667,312 |
| | 1H-imidazo[4,5-c]quinolin-4-amine | Example 56 |
| IRM 7 | N-{2-[4-amino-2-(ethoxymethyl)-1H- | U.S. Pat. No. 6,541,485^{#} |
| | imidazo[4,5-c]quinolin-1-yl]ethyl}-N′- | |
| | isopropylurea | |
| IRM 8 | N-(2-{2-[4-amino-2-(ethoxymethyl)-1H- | U.S. Pat. No. 6,660,735 |
| | imidazo[4,5-c]quinolin-1-yl]ethoxy}ethyl)-N′- | Example 53 |
| | phenylurea | |
| IRM 9 | 1-[2-(pyridin-4-ylmethoxy)ethyl]-1H- | U.S. Pat. No. 6,664,260 |
| | imidazo[4,5-c]quinolin-4-amine | Example 15 |
| IRM 10 | 2-butyl-1-[3-(methylsulfonyl)propyl]-1H- | U.S. Pat. No. 6,664,264 |
| | imidazo[4,5-c]quinoline-4-amine | Example 19 |

^{#}This compound is not specifically exemplified but can be readily prepared using the synthetic methods disclosed in the cited reference.

Cream formulations were used in several of the examples. The composition of the creams is shown in the table below where the amounts are % w/w. The formulations were prepared using the methods described in WO 03/045391.

| Component | 1% IRM 2 Cream | 5% IRM 3 Cream |
|---|---|---|
| IRM | 1.00 | 5.0 |
| Isostearic acid | 6.05 | 50.0 |
| Isopropyl myristate | 8.95 | — |
| ¹CARBOPOL 974P | 1.00 | 1.0 |
| Water | 64.55 | 30.6 |
| Disodium EDTA | 0.05 | 0.05 |
| Poloxamer 188 | 2.5 | 2.5 |
| Propylene glycol | 15 | 10.0 |
| Methylparaben | 0.2 | 0.2 |
| 20% sodium hydroxide solution | 0.7 | 0.7 |

¹Available from Noveon, Cleveland, Ohio

### Example 1

Devices were prepared by forming approximately 0.02 g of cotton (sterile cotton balls available from Walgreen Co., Deerfield, Ill. as ITEM 666504 WGPS 130WCU-1) into a cylindrical shape and then tying a silk suture around one end. A solution containing 1.0% by weight of IRM 1 in isostearic acid was prepared. The devices were saturated with either the IRM 1 solution or with isostearic acid (vehicle). The devices were removed at the end of the treatment period by pulling on the silk suture. Two groups of 3 rats were dosed intravaginally with devices containing the IRM 1 solution. In one group the devices were removed after two hours; in the second group the devices were removed after 4 hours. A third group was dosed with devices containing isostearic acid. The vaginal tissue and serum TNF and MCP-1 levels for all three groups were determined at 4 hours post dosing. The results are shown in the table below where each value is the mean of the values for the 3 rats in the group.

| Cytokine Concentrations | |
|---|---|
| at 4 Hours Post Dosing | |
| TNF (pg/mL) | MCP-1 (pg/mL) |

| Treatment | Serum | Tissue | Serum | Tissue |
|---|---|---|---|---|
| Vehicle/device | 0 | 33 | 124 | 408 |
| IRM 1/device | 0 | 328 | 122 | 961 |
| 2 hr | | | | |
| IRM 1/device | 15 | 452 | 93 | 894 |
| 4 hr | | | | |

### Example 2

Devices were prepared as described in Example 1 and saturated with either a solution containing 1.0% by weight of IRM 1 in isostearic acid or with a solution containing 0.1% by weight of IRM 1 in isostearic acid. Rats were dosed intravaginally; the devices were removed after 2 hours. Cytokines were assayed at 2, 4, and 6 hours post dosing. A group of rats that did not receive any treatment served as controls. The results are shown in the table below where each value is the mean of the values for 3 rats.

| Time | | | | | |
|---|---|---|---|---|---|
| (hours) | Treatment | Cytokine Concentrations | | | |
| post | IRM 1 | TNF (pg/mL) | | MCP-1 (pg/mL) | |
| dosing | device | Serum | Tissue | Serum | Tissue |
| 2 hr | 0.1% | 0 | 58 | 146 | 69 |
| 2 hr | 1.0% | 0 | 461 | 120 | 247 |
| 4 hr | 0.1% | 0 | 136 | 155 | 252 |
| 4 hr | 1.0% | 1 | 1427 | 123 | 649 |
| 6 hr | 0.1% | 0 | 215 | 128 | 137 |
| 6 hr | 1.0% | 3 | 161 | 279 | 484 |
| 2 hr | Controls | 0 | 76 | 113 | 108 |

### Example 3

Devices were prepared as described in Example 1 and saturated with either a solution containing 1.0% by weight of IRM 1 in isostearic acid (ISA) or with a solution containing 1.0% by weight of IRM 1 in 50/50 w/w isostearic acid (ISA)/isopropyl myristate (IPM). Rats were dosed intravaginally; the devices were removed after 2 hours. Cytokines were assayed at 4 hours post dosing. The results are shown in the table below where each value is the mean of the values for 3 rats.

| | Cytokine Concentrations | |
|---|---|---|
| | at 4 Hours Post Dosing | |
| Treatment | TNF (pg/mL) | MCP-1 (pg/mL) |

| IRM 1/device | Serum | Tissue | Serum | Tissue |
|---|---|---|---|---|
| ISA solution | 7 | 571 | 101 | 583 |
| ISA/IPM | 0 | 263 | 113 | 686 |
| solution | | | | |

### Example 4

Devices were prepared as described in Example 1 and saturated with either a solution containing 1.0% by weight of IRM 2 in 50/50 w/w isostearic acid (ISA)/isopropyl myristate (IPM) or with 50/50 w/w ISA/IPM (vehicle). Rats were dosed intravaginally; the devices were removed after 15 minutes, 30 minutes, 60 minutes or 120 minuets. One group of rats was dosed with 1% IRM 2 cream. The cream formulation was not removed. Cytokines were assayed at 4 hours post dosing. The results are shown in the table below where each value is the mean of the values for 5 rats.

| | Cytokine Concentrations | | | |
|---|---|---|---|---|
| | at 4 Hours Post Dosing | | | |
| | TNF (pg/mL) | | MCP-1 (pg/mL) | |
| Treatment | Serum | Tissue | Serum | Tissue |
| IRM 2/device 15 min | 0 | 493 | 68 | 433 |
| IRM 2/device 30 min | 0 | 390 | 83 | 454 |
| IRM 2/device 60 min | 0 | 537 | 118 | 889 |
| IRM 2/device 120 min | 0 | 716 | 92 | 2462 |
| Vehicle/device 120 min | 0 | 443 | 73 | 63 |
| 1% IRM 2 cream | 92 | 1691 | 94 | 2175 |

### Example 5

Devices were prepared from either cotton as described in Example 1 or from polyurethane foam (Medisorb 100—1.25:Polysorbate 60 at 1% concentration at 1.25/1 ratio, from Lendell Manufacturing, Inc, St. Charles, Mich.). The devices were saturated with one of the following solutions: 0.1% ERM 3 in 50/50 ISA/IPM; 1.0% IRM 3 in 50/50 ISA/IPM; 3.0% IRM 3 in 50/50 ISA/IPM or with 50/50 ISA/IPM (vehicle). Rats were dosed intravaginally; the devices were removed after 2 hours. A group of rats that did not receive any treatment served as controls. Cytokines were assayed at 4 hours post dosing. The results are shown in the table below where each value is the mean of the values for 3 rats.

| Cytokine Concentrations | |
|---|---|
| at 4 Hours Post Dosing | |
| TNF (pg/mL) | MCP-1 (pg/mL) |

| Treatment | Serum | Tissue | Serum | Tissue |
|---|---|---|---|---|
| 0.1% IRM 3/cotton | 0 | 108 | 72 | 179 |
| 0.1% IRM 3/foam | 0 | 85 | 77 | 143 |
| 1.0% IRM 3/cotton | 0 | 173 | 111 | 468 |
| 1.0% IRM 3/foam | 0 | 148 | 86 | 279 |
| 3.0% IRM 3/cotton | 0 | 175 | 79 | 402 |
| 3.0% IRM 3/foam | 0 | 302 | 105 | 351 |
| Vehicle/cotton | 0 | 97 | 49 | 101 |
| Vehicle/foam | 0 | 57 | 98 | 94 |
| Controls | 0 | 139 | 81 | 27 |

### Example 6

Devices were prepared from cotton pellets (cotton pellets, non-sterile, 100% cotton, size #3, 5/32 inch (0.4 cm); available from Richmond Dental, a division of Barnhardt Manufacturing, Charlotte, N.C.). The devices were saturated with one of the following solutions: 1.0% IRM 2 in 50/50 ISA/IPM; 1.0% IRM 4 in 50/50 ISA/IPM; 1.0% IRM 5 in 50/50 ISA/IPM; 1.0% IRM 6 in 50/50 ISA/IPM; 1.0% IRM 7 in 50/50 ISA/IPM; 1.0% IRM 8 in 50/50 ISA/IPM; or with 50/50 ISAAIPM (vehicle). Rats were dosed intravaginally; the devices were removed after 2 hours. One group of rats was dosed with 1% IRM 2 cream. Cytokines were assayed at 4 hours post dosing. The results are shown in the table below where each value is the mean of the values for 3 rats.

| Cytokine Concentrations | |
|---|---|
| at 4 Hours Post Dosing | |
| TNF (pg/mL) | MCP-1 (pg/mL) |

| Treatment | Serum | Tissue | Serum | Tissue |
|---|---|---|---|---|
| 1% IRM 2/device | 4 | 384 | 114 | 1016 |
| 1% IRM 4/device | 0 | 109 | 105 | 713 |
| 1% IRM 5/device | 1 | 358 | 108 | 958 |
| 1% IRM 6/device | 1 | 491 | 114 | 1840 |
| 1% IRM 7/device | 0 | 219 | 93 | 642 |
| 1% IRM 8/device | 0 | 294 | 82 | 331 |
| Vehicle/device | 1 | 143 | 79 | 272 |
| 1% IRM 2 Cream | 176 | 725 | 365 | 1570 |

### Example 7

Devices were prepared from cotton pellets as described in Example 6. The devices were saturated with one of the following solutions: 5.0% IRM 3 in 50/50 ISA/IPM; 5.0% IRM 7 in 50/50 ISA/IPM; 5.0% IRM 9 in 50/50 ISA/IPM; 5.0% IRM 10 in 50/50 ISA/IPM; or with 50/50 ISA/IPM (vehicle). Rats were dosed intravaginally; the devices were removed after 2 hours. Cytokines were assayed at 2, 4, and 6 hours post dosing. The results are shown in the table below where each value is the mean of the values for 6 rats.

| Time | | Cytokine Concentrations | | | |
|---|---|---|---|---|---|
| (hours) | | TNF (pg/mL) | | MCP-1 (pg/mL) | |
| post dose | Formulation | Serum | Tissue | Serum | Tissue |
| 2 hr | 5% IRM 3 | 4 | 809 | 95 | 815 |
| 2 hr | 5% IRM 7 | 1 | 512 | 92 | 498 |
| 2 hr | 5% IRM 9 | 30 | 597 | 85 | 328 |
| 2 hr | 5% IRM 10 | 16 | 1110 | 111 | 739 |
| 4 hr | 5% IRM 3 | 3 | 608 | 114 | 1260 |
| 4 hr | 5% IRM 7 | 0 | 460 | 112 | 851 |
| 4 hr | 5% IRM 9 | 4 | 697 | 131 | 1556 |
| 4 hr | 5% IRM 10 | 25 | 887 | 160 | 1440 |
| 6 hr | 5% IRM 3 | 5 | 114 | 171 | 840 |
| 6 hr | 5% IRM 7 | 2 | 267 | 140 | 670 |
| 6 hr | 5% IRM 9 | 8 | 248 | 180 | 850 |
| 6 hr | 5% IRM 10 | 10 | 519 | 155 | 975 |
| 4 hr | Vehicle | 4 | 48 | 115 | 130 |

### Example 8

Cotton devices were prepared as described in Example 1. The devices were saturated with either a solution containing 1% by weight of IRM 2 in 50/50 w/w isostearic acid/isopropyl myristate or with 50/50 w/w isostearic acid/isopropyl myristate (vehicle). Three groups of rats were dosed intravaginally 2 times a week for 3 weeks (Tuesday, Friday, Monday, Thursday, Monday, Thursday) with 1% IRM 2 device, vehicle device or with 1% IRM 2 cream. The devices were removed after 2 hours. The cream was left in place. Cytokines were assayed 4 hours post dosing of the final dose. Three more groups of rats were dosed intravaginally with 1% IRM 2 device, vehicle device or with 1% ERM 2 cream. The devices were removed after 2 hours. The cream was left in place. Cytokines were assayed 4 hours post dosing. A group of rats that did not receive any treatment served as controls. The results are shown in the table below where each value is the mean value for 3 rats.

| | Cytokine Concentration | | | |
|---|---|---|---|---|
| | at 4 Hours Post Dosing | | | |
| | TNF (pg/mL) | | MCP-1 (pg/mL) | |
| Treatment | Serum | Tissue | Serum | Tissue |
| IRM 2 device - single | 0 | 888 | 59 | 1390 |
| IRM 2 device - multiple | 0 | 1075 | 87 | 2353 |
| Vehicle device - single | 0 | 291 | 43 | 59 |
| Vehicle device - | 0 | 279 | 28 | 150 |
| multiple | | | | |
| IRM 2 cream - single | 27 | 991 | 86 | 1720 |
| IRM 2 cream - multiple | 8 | 264 | 66 | 768 |
| Controls | 0 | 117 | 51 | 36 |

### Example 9

Groups of 3 rats were treated as described in Example 5 and necropsied 22 hours after the devices were removed. Vaginas and vulvas were collected, fixed and processed routinely for histologic examination. The results are summarized in the table below. Inflammation was scored as follows: 0=none, 1=minimal, 2=mild, 3=moderate, 4=severe. The values in the tables are the mean of the scores for 3 rats. The value for erosion or ulceration is expressed as an incidence, for example 0/3 means that none of the 3 rats in that particular group showed erosion or ulceration.

| | | Treatment Solution | | | |
|---|---|---|---|---|---|
| | | | 0.1% | 1.0% | 3.0% |
| Tissue | Device | Vehicle | IRM 3 | IRM 3 | IRM 3 |
| Vagina | cotton | 0.67 | 1.0 | 2.5 | 3.17 |
| Inflammation | foam | 0.83 | 2.17 | 2.83 | 2.5 |
| Vagina | cotton | 0/3 | 0/3 | 0/3 | 0/3 |
| Erosion or | foam | 0/3 | 0/3 | 0/3 | 0/3 |
| ulceration | | | | | |
| Vulva | cotton | 0.5 | 0.33 | 0.5 | 2.17 |
| Inflammation | foam | 0.33 | 0.33 | 0.33 | 1.75 |
| Vulva | cotton | 0/3 | 0/3 | 0/2* | 0/3 |
| Erosion or | foam | 0/3 | 0/3 | 0/3 | 0/2* |
| ulceration | | | | | |

*Tissue from 1 rat in the group was not assessable.

### Example 10

Cotton devices were prepared as described in Example 1. The devices were saturated with a solution containing 5% by weight of IRM 3 in 50/50 w/w isostearic acid/isopropyl myristate. One group of 5 rats was dosed intravaginally with the devices. The devices were removed after 2 hours. A second group of 5 rats was dosed intravaginally with 5% IRM 3 cream. The cream was washed out after 2 hours. A third group of 5 rats was dosed intravaginally with 5% IRM 3 cream but the cream was not removed. The rats were necropsied 24 hours after treatment initiation. Vaginas and vulvas were collected, fixed and processed routinely for histologic examination. The results are summarized in the table below. The scoring system described in Example 9 was used.

| | Treatment | | |
|---|---|---|---|
| | 5% IRM | 5% IRM 3 cream | 5% IRM 3 cream |
| Tissue | 3/device | washed out | not removed |
| Vagina - Inflammation | 3.1 | 3.0 | 3.0 |
| Vagina - erosion | 0/5 | 3/5 | 3/5 |
| Vagina - ulceration | 0/5 | 1/5 | 1/5 |
| Vulva - Inflammation | 1.7 | 2.1 | 1.6 |
| Vulva - preulcer | 3/5 | 1/5 | 2/5 |
| Vulva - ulceration | 0/5 | 0/5 | 1/5 |

### Example 11

Groups of 3 rats were treated as described in Example 8 and necropsied 22 hours after the devices were removed. Uterus, cervix, vagina, vulva and perineal skin were collected, fixed and processed routinely for histologic examination. The results are summarized in the table below.

| | | Treatment group/Lesion incidence | | | | | |
|---|---|---|---|---|---|---|---|
| | | Vehicle/ | | 1% IRM 2/ | | 1% IRM 2 | |
| | | Device | | Device | | Cream | |
| | | Multiple | Single | Multiple | Single | Multiple | Single |
| Site | Lesion | dose | dose | dose | dose | dose | dose |
| Vulva | Edema, | 0/3 | 0/3 | 1/3 | 0/3 | 3/3 | 0/3 |
| | lamina | | | | | | |
| | propria | | | | | | |
| | Inflammation, | 0/3 | 0/3 | 3/3 | 3/3 | 3/3 | 0/3 |
| | lamina | | | | | | |
| | propria | | | | | | |
| | Spongiosis, | 0/3 | 0/3 | 1/3 | 0/3 | 3/3 | 0/3 |
| | epithelium | | | | | | |
| | Necrosis, | 0/3 | 0/3 | 1/3 | 1/3 | 0/3 | 0/3 |
| | epithelium | | | | | | |
| | Intraepithelial | 0/3 | 0/3 | 0/3 | 1/3 | 1/3 | 0/3 |
| | pustules | | | | | | |
| | Erosion | 0/3 | 0/3 | 0/3 | 0/3 | 2/3 | 0/3 |
| | Ulceration | 0/3 | 0/3 | 0/3 | 0/3 | 3/3 | 0/3 |
| Vagina | Edema, | 0/3 | 0/3 | 0/3 | 0/3 | 3/3 | 0/3 |
| | lamina | | | | | | |
| | propria | | | | | | |
| | Inflammation, | 1/3 | 0/3 | 3/3 | 3/3 | 3/3 | 0/3 |
| | lamina | | | | | | |
| | propria | | | | | | |
| Cervix | Inflammation | 0/3 | 0/3 | 1/3 | 0/3 | 2/3 | 0/3 |
| | Cavitation | 0/3 | 0/3 | 0/3 | 0/3 | 1/3 | 0/3 |
| | (epithelium) | | | | | | |
| Perineal | Exudate on | 0/3 | 0/3 | 0/3 | 0/3 | 1/3 | 0/3 |
| skin | surface, | | | | | | |
| | epidermis | | | | | | |
| | Inflammation, | 0/3 | 0/3 | 1/3 | 1/3 | 2/3 | 0/3 |
| | superficial | | | | | | |
| | dermis | | | | | | |
| | Subcorneal | 0/3 | 0/3 | 1/3 | 0/3 | 0/3 | 0/3 |
| | pustules, | | | | | | |
| | epidermis | | | | | | |
| | Spongiosis, | 0/3 | 0/3 | 1/3 | 0/3 | 1/3 | 1/3 |
| | epidermis | | | | | | |

The complete disclosures of the patents, patent documents and publications cited herein are incorporated by reference in their entirety as if each were individually incorporated. In case of conflict, the present specification, including definitions, shall control. Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A method of delivering an immune response modifier (IRM) compound to a mucosal surface so as to achieve immunomodulation with reduced irritation, comprising:
interrupted delivery of an IRM compound other than imiquimod by intermittently applying the IRM to the mucosal surface and, after each application, removing from the mucosal surface a substantial amount of the IRM at a time before it would otherwise be naturally absorbed or eliminated.

2. The method of claim 1 wherein the IRM is applied and removed with the same device.

3. The method of claims 1 or 2 wherein the mucosal surface is associated with a condition selected from the group consisting of a cervical dysplasia, a papilloma virus infection of the cervix, a low-grade squamous intraepithelial lesion, a high-grade squamous intraepithelial lesion, atypical squamous cells of undetermined significance, a cervical intraepithelial neoplasia, an atopic allergic response, allergic rhinitis, a neoplastic lesion, and a premalignant lesion.

4. The method of claim 3 wherein the mucosal surface is on the cervix and the associated condition is selected from the group consisting of cervical dysplasia high-grade squamous intraepithelial lesions, low-grade squamous intraepithelial lesions, and atypical squamous cells of undetermined significance with the presence of high risk HPV.

5. The method of claim 4 wherein the mucosal surface is on the cervix and the associated condition is atypical squamous cells of undetermined significance with the presence of high risk HPV.

6. The method of claim 3 wherein the mucosal surface is on the cervix and the associated condition is a papilloma virus infection of the cervix.

7. The method of any one of claims 1 through 6 wherein the IRM is applied to the mucosal surface using a device selected from the group consisting of a tampon, a cervical cap, a diaphragm, a cotton swab, a cotton sponge, a foam sponge, and a suppository.

8. The method of claim 1, wherein a substantial amount of the IRM is removed less than 8 hours after it is applied.

9.-10. (canceled)

11. The method of claim 1 wherein a substantial amount of the IRM is removed 2 hours or less after it is applied.

12.-13. (canceled)

14. The method of claim 1 wherein the IRM activates the TLR selected from the group consisting of TLR6, TLR7, TLR8, TLR 9, and combinations thereof.

15.-16. (canceled)

17. The method of claim 1 wherein the IRM is selected from the group consisting of imidazoquinoline amines, tetrahydroimidazoquinoline amines, imidazopyridine amines, 6,7-fused cycloalkylimidazopyridine amines, 1,2-bridges imidazoquinoline amines, imidazonaphthyridine amines, imidazotetrahydronaphthyridine amines, oxazoloquinoline amines, thiazoloquinoline amines, oxazolopyridine amines, thiazolopyridine amines, oxazolonaphthyridine amines, thiazolonaphthyridine amines, 1H-imidazo dimers fused to pyridine amines, quinoline amines, tetrahydroquinoline amines, naphthyridine amines, or tetrahydronaphthyridine amines, pharmaceutically acceptable salts thereof, and combinations thereof.

18.-19. (canceled)

20. The method of claim 17 wherein the IRM is an imidazonaphthyridine amine or a pharmaceutically acceptable salt thereof.

21. The method of claim 20 wherein the IRM is 1-(2-methylpropyl)-1H-imidazo[4,5-c][1,5]naphthyridin-4-amine or a pharmaceutically acceptable salt thereof.

22. The method of claim 1 wherein the IRM comprises a 2-aminopyridine fused to a five membered nitrogen-containing heterocyclic ring.

23.-26. (canceled)

27. A method of treating a condition associated with a mucosal surface with an immune response modifier (IRM) compound and reducing irritation caused by the IRM, comprising:
interrupted delivery of an IRM other than imiquimod by intermittently applying the IRM to the affected mucosal surface for a time sufficient to achieve therapeutic immunomodulation and, after each application, removing from the mucosal surface a substantial amount of the IRM at a time before it would otherwise be naturally absorbed or eliminated.

28.-33. (canceled)

34. The method of claim 27 wherein the IRM is predispersed within a solid matrix capable of releasing the IRM while in contact with the mucosal surface.

35. (canceled)

36. The method of claim 34 wherein the solid matrix is selected from the group consisting of a tampon, a sponge, and a suppository.

37.-40. (canceled)
